(19) United States
(12) Patent Application Publication
(10) Pub. No.: US 2017/0209413 A1
(43) Pub. Date: Jul. 27, 2017

(54) USE OF ASPARAGUSIC ACID AND DERIVATIVES THEREOF TO TREAT SYMPTOMS AND DISORDERS OF NEUROLOGICAL DISEASE

(71) Applicant: Joseph WREEN, St. Helena Island, SC (US)

(72) Inventor: Joseph WREEN, St. Helena Island, SC (US)

(21) Appl. No.: 15/416,450

(22) Filed: Jan. 26, 2017

Related U.S. Application Data
(60) Provisional application No. 62/287,171, filed on Jan. 26, 2016.

(51) Int. Cl. A61K 31/385; A61K 9/00; A61K 36/88

(52) U.S. Cl. CPC A61K 31/385; A61K 36/88; A61K 9/0053

(57) ABSTRACT

Embodiments disclosed herein provide methods for treating and/or ameliorating symptoms of certain neurological diseases. The methods encompass administration of compositions comprising 1,2-dithiolane-4-carboxylic acid (asparagusic acid) and derivatives and condensation products thereof.

### CROSS REFERENCE TO RELATED APPLICATION

This application claims the benefit of U.S. Provisional Application No. 62/287,171 filed on Jan. 26, 2016. The entire contents of the above-identified priority application is hereby fully incorporated herein by reference.

### TECHNICAL FIELD

The subject matter disclosed herein is generally directed to methods of treating and/or ameliorating symptoms of certain neurological diseases using asparagusic acid and derivatives thereof.

### BACKGROUND

In recent years it has been shown that copper plays an important role in the health of the human brain. Improper copper oxidation has been linked to several neurological disorders including Alzheimers, Parkinson's, Menkes' and Wilson's disease. (10, 11, 12). Copper has also been identified as a critical cofactor in enzymes that activates the brain's neurotransmitters in response to stimuli. A recent study by researchers at the U.S. Department of Energy's Lawerence Berkeley National Laboratory has shown that proper copper levels are also essential to the health of the brain at rest. (12). Traditionally, copper has been regarded as a static metabolic cofactor that must be buried in enzymes to protect against the generation of reactive oxygen species and subsequent free radical damage. However, it has also been shown that dynamic and loosely bound pools of copper can modulate neural activity and are essential for the normal development of synapses and circuits. (13). Further, many proteins involved in carrying and transporting copper into the cell use a copper-sulfur linkage via the amino acid cysteine or methionine. (4, 14, 15, 16). Therefore, what are needed are compositions that may be used to maintain proper copper levels and/or sulfur levels in the brain in order to sustain normal and healthy brain function.

### SUMMARY

In certain aspects described herein, a method for treating a disease characterized by improper copper and/or sulfur concentration in nerve cells of the central nervous system comprises administering to a subject in need thereof an effective amount of a composition comprising asparagusic acid, derivatives of asparagusic acid, condensation products of asparagusic acid or its derivatives, or a combination thereof. In certain example embodiments, the subject suffers from nerve cell malfunction and/or death in the substantia nigra part of the brain. In certain other aspects, the subject suffers from Parkinson's disease. In certain example embodiments, the condensation product comprises asparagusic acid or a derivative therefor and an amino acid, such as arginine. In certain example embodiments, the condensation product is asparaptine.

In another aspect described herein, a method for restoring impaired motor function associated with neurological diseases comprises administering to a subject in need thereof an effective amount of a composition comprising asparagusic acid, derivatives of asparagusic acid, condensation products of asparagusic acid or its derivatives, or a combination thereof. In certain example embodiments, the subject suffers from one or more of tremor, bradykinesia, rigidity, and postural instability. In certain example embodiments, the subject suffers from Parkinson's disease. In one example embodiment, the composition comprises asparagusic acid. In certain example embodiments, the condensation product comprises asparagusic acid, or a derivative therefor, and an amino acid, such as arginine. In certain example embodiments, the condensation product is asparaptine.

In yet another aspect descried herein, a method for increasing copper and/or sulfur concentration in nerve cells and/or restoring or maintaining proper copper and/or sulfur homeostasis in nerve cells comprises administering to a subject in need thereof an effective amount of a composition comprising asparagusic acid, derivatives of asparagusic acid, condensation products of asparagusic acid or derivatives of asparagusic acid, or combinations thereof. In certain example embodiments, the subject suffers from one or more of tremor, bradykinesia, rigidity, and postural instability. In certain example embodiments, the subject suffers from Parkinson's disease. In one example embodiment, the composition comprises asparagusic acid. In certain example embodiments, the condensation product comprises asparagusic acid, or a derivative therefor, and an amino acid, such as arginine. In certain example embodiments, the condensation product is asparaptine.

These and other aspects, objects, features, and advantages of the example embodiments will become apparent to those having ordinary skill in the art upon consideration of the following detailed description of illustrated example embodiments.

### DETAILED DESCRIPTION OF THE EXAMPLE EMBODIMENTS

### Overview

Embodiments disclosed herein provide methods for treating and/or ameliorating symptoms of certain neurological diseases. The methods encompass administration of compositions comprising 1,2-dithiolane-4-carboxylic acid (asparagusic acid) and derivatives and condensation products thereof. While not being limited to the following hypothesis, Applicant believes that asparagusic acid and its derivatives may act as a reducing agent of copper, reducing Cu⁺² to Cu⁺¹. In order to enter nerve cells, coppers must be in the Cu⁺¹ state (5, 14). As noted above, improper copper balance both inside and outside the cell is known to contribute to a number of neurological diseases and disorders. In addition to maintaining proper copper homeostasis, asparagusic acid and its derivatives may also help regulate and maintain proper sulfur concentrations in nerve cells.

As used herein, “treatment” or “treating” includes both a complete restoration of normal neurological function and/or cessation of nerve cell death or malfunction as well as amelioration in the severity or frequency of symptoms associated with the disease or disorder

### Restoring and Maintaining Proper Copper and Sulfur Concentrations

Thus in one aspect, the present application provides a method of increasing copper and/or sulfur concentration in nerve cells and/or restoring or maintaining proper copper and/or sulfur homeostasis in nerve cells, comprising administering to a subject in need thereof an effective amount of a composition comprising one or more compounds selected from the following:

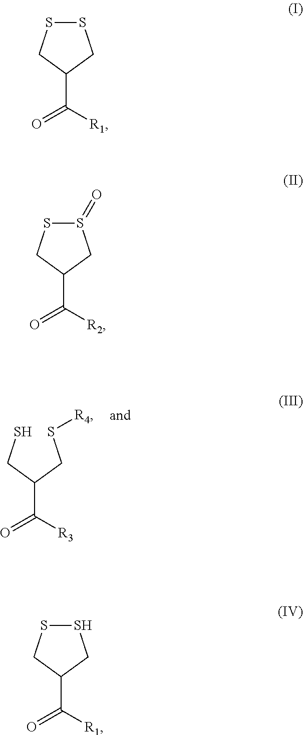

wherein R1 is H, CH₃, OH, or OCH₃, R2 is H, CH₃, OH, or OCH₃, R3 is H, CH₃, OH, or O CH₃ and, R4 is H, CH₃, OCH.

In certain example embodiments, the composition comprises asparagusic acid, asparagusic acid methyl ester, asparagusic ethyl ester, asparagusic acid glucoside; asparagusic acid S-oxide, asparagusic acid S-oxide methyl ester, dihydroasparagusic acid, or S-acetyldihydroasparagusic acid, or a combination thereof. In one example embodiment, the compound is asparagusic acid.

The compounds of formulas (I)-(IV) may be in the form of a condensation product. In certain example embodiments, the condensation product results from condensation of one of the compounds of formulas (I) to (IV) with an amino acid. In certain example embodiments, the amino acid is arginine. In certain other example embodiments, the condensation product is asparaptine as represented by formula (V) below.

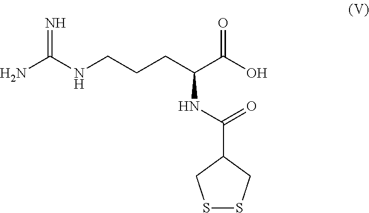 [SMILES: N=C(N)NCCC[C@H](NC(=O)C1CSSC1)C(=O)O]

Therefore, in certain example embodiments the composition may comprise one or more condensation products of the compounds of formula (I) to (IV), or a mixture of the compounds of formula (I) to (IV) and condensation products thereof.

### Restoring Motor Function

Some neurological diseases and disorders have symptoms that manifest as reduction or loss of normal motor function. Administration of the compounds disclosed herein may reduce the severity or frequency of motor dysfunction and/or restore normal motor function. Therefore, in another example embodiment, the present application provides methods of restoring impaired motor function associated with neurological diseases comprising administering to a subject in need thereof a composition comprising one or more of the compounds of formulas (I) to (IV), one or more condensation products of the compounds of formulas (I) to (IV), or a mixture of the one or more of the compounds of formulas (I) to (IV) and one or more condensation products of the compounds of formulas (I) to (IV).

In certain example embodiments, the composition comprises asparagusic acid, asparagusic acid methyl ester, asparagusic ethyl ester, asparagusic acid glucoside; asparagusic acid S-oxide, asparagusic acid S-oxide methyl ester, dihydroasparagusic acid, or S-acetyldihydroasparagusic acid, or a combination thereof. In one example embodiment, the compound is asparagusic acid.

In certain example embodiments, the composition comprises a condensation product of one of more of the compounds of formula (I) to (IV). In certain example embodiments, the condensation product results from the condensation of one of the compounds of formulas (I) to (IV) with an amino acid. In certain example embodiments, the amino acid is arginine. In certain example embodiments, the condensation product is asparaptine.

Therefore, in certain example embodiments the composition may comprise one or more condensation products of the compounds of formula (I) to (IV), or a mixture of the compounds of formula (I) to (IV) and condensation products thereof.

### Treatment of Neurological Diseases

As noted above, proper copper levels in nerve cells of the central nervous system play a role in the development and progression of certain neurological diseases. The present composition may help remove excess copper from the central nervous system by allowing normal uptake of copper into nerve cells. Therefore, in another example embodiment, the present application provides methods for treating a diseases characterized by improper copper and/or sulfur concentrations in the central nervous system comprising administering to a subject in need thereof, a composition comprising one or more of the compounds of formulas (I) to (IV), one or more condensation products of the compounds of formulas (I) to (IV), or a mixture of one or more of the compounds of formulas (I) to (IV) and one or more condensation products of the compounds of formulas (I) to (IV).

In certain example embodiments, the composition comprises asparagusic acid, asparagusic acid methyl ester, asparagusic ethyl ester, asparagusic acid glucoside; asparagusic acid S-oxide, asparagusic acid S-oxide methyl ester, dihydroasparagusic acid, or S-acetyldihydroasparagusic acid, or a combination thereof. In one example embodiment, the compound is asparagusic acid.

In certain example embodiments, the composition comprises a condensation product of one or more of the compounds of formula (I) to (IV). In certain example embodiments, the condensation product results from the condensation of one of the compounds of formulas (I) to (IV) with an amino acid. In certain example embodiments, the amino acid is arginine. In certain example embodiments, the condensation product is asparaptine.

In certain example embodiments, the composition comprises a condensation product of one of the compounds of formula (I) to (IV). In certain example embodiments, the condensation product results from the condensation of one of the compounds of formulas (I) to (IV) with an amino acid. In certain example embodiments, the amino acid is arginine. In certain example embodiments, the condensation product is asparaptine.

Therefore, in certain example embodiments the composition may comprise one or more condensation products of the compounds of formula (I) to (IV), or a mixture of the compounds of formula (I) to (IV) and condensation products thereof.

In certain example embodiments, the subject has a disease characterized by excess accumulation of copper in the cerebrospinal fluid of the central nervous system. In certain other example embodiments, the subject has a disease or disorder characterized by cell death or malfunction in the brain. In another example embodiment, the subject has a disease or disorder characterized by cell death or malfunction in the substantia nigra region of the brain. In certain other example embodiments, the subject suffers from Parkinson's disease.

### Pharmaceutical Formulations

The compounds and condensation products thereof used in the compositions of the present invention may be synthesized using methods known in the art. In certain example embodiments, the compounds and/or condensation products thereof may be isolated directly from Asparagus officinalis. One example embodiment for isolating the compounds and/or condensation products thereof directly from A. officinalis is described in the Example section below.

The compositions described herein can be provided as physiologically acceptable formulations using known techniques, and the formulations can be administered by standard routes. 100% pure isomers are contemplated by this invention; however a stereochemical isomer (labeled as α or β, or as R or S) may be a mixture of both in any ratio, where it is chemically possible by one skilled in the art. Also contemplated by this invention are both classical and non-classical bioisosteric atom and substituent replacements, such as are described by Patani and Lavoie (“Bio-isosterism: a rational approach in drug design” Chem. Rev. (1996) p. 3147-3176) and are well known to one skilled in the art. Such bioisosteric replacements include, for example, but are not limited to, substitution of a S or a NH for an O.

The formulations in accordance with the present invention can be administered in the form of a tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, an eye drop or a transdermal patch.

The formulations include those suitable for oral, rectal, nasal, inhalation, topical (including dermal, transdermal, buccal, and eye drops), vaginal, parenteral (including subcutaneous, intramuscular, intravenous, intradermal, intraocular, intratracheal, and epidural) or inhalation administration. In one exemplary embodiment, the corticosteroid pro-drugs of the present invention are formulated for transcleral delivery. Transcleral delivery includes subconjunctival, subtenon, and retrobulbar trancleral delivery. In one exemplary embodiment, the NSAID pro-drugs are formulated for administration topically as eye drops. The formulations can conveniently be presented in unit dosage form and can be prepared by conventional pharmaceutical techniques. Such techniques include the step of bringing into association the active ingredient and a pharmaceutical carrier(s) or excipient(s). In general, the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil emulsion, etc.

A tablet may be made by compression or molding, optionally with one or more accessory ingredients. Compressed tablets may be prepared by compressing, in a suitable machine, the active ingredient in a free-flowing form such as a powder or granules, optionally mixed with a binder, lubricant, inert diluent, preservative, surface-active or dispersing agent. Molded tablets may be made by molding, in a suitable machine, a mixture of the powdered compound moistened with an inert liquid diluent. The tablets may optionally be coated or scored and may be formulated so as to provide a slow or controlled release of the active ingredient therein.

Formulations suitable for topical administration in the mouth include lozenges comprising the ingredients in a flavored base, usually sucrose and acacia or tragacanth; pastilles comprising the active ingredient in an inert base such as gelatin and glycerin, or sucrose and acacia; and mouthwashes comprising the ingredient to be administered in a suitable liquid carrier.

Formulations suitable for topical administration to the skin may be presented as ointments, creams, gels, pastes, and eye drops comprising the ingredient to be administered in a pharmaceutical acceptable carrier.

Formulations for rectal administration may be presented as a suppository with a suitable base comprising, for example, cocoa butter or a salicylate.

Formulations suitable for nasal administration, wherein the carrier is a solid, include a coarse powder having a particle size, for example, in the range of 20 to 500 microns which is administered in the manner in which snuff is taken; i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration may be presented as pessaries, tampons, creams, gels, pastes, foams or spray formulations containing, in addition to the active ingredient, ingredients such as carriers as are known in the art to be appropriate.

Formulation suitable for inhalation may be presented as mists, dusts, powders or spray formulations containing, in addition to the active ingredient, ingredients such as carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Formulations suitable for parenteral administration also include, but are not limited to, nanoparticle formulations made by numerous methods as disclosed in U.S. patent application Ser. No. 10/392,403 (Publication No. US 2004/0033267), U.S. patent application Ser. No. 10/412,669 (Publication No. US 2003/0219490), U.S. Pat. No. 5,494,683, U.S. patent application Ser. No. 10/878,623 (Publication No. US 2005/0008707), U.S. Pat. No. 5,510,118, U.S. Pat. No. 5,524,270, U.S. Pat. No. 5,145,684, U.S. Pat. No. 5,399,363, U.S. Pat. No. 5,518,187, U.S. Pat. No. 5,862,999, U.S. Pat. No. 5,718,388, and U.S. Pat. No. 6,267,989, all of which are hereby incorporated herein by reference in there entirety. A review of drug formulation technology is provided in “Water Insoluble Drug Formulation” by Rong Liu, editor, pp. 1-633, (2000) CRC Press LLC, which is incorporated herein by reference in its entirety.

It should be understood that, in addition to the ingredients particularly mentioned above, the formulations of the present invention may include other agents conventional in the art having regard to the type of formulation in question, for example, those suitable for oral administration may include flavoring agents, and nanoparticle formulations (e.g.; less than 2000 nanometers, preferably less than 1000 nanometers, most preferably less than 500 nanometers in average cross section) may include one or more than one excipient chosen to prevent particle agglomeration.

In certain example embodiments, the composition is formulated for oral administration.

### Dosage and Administration

In certain example embodiments, the composition is formulated so that the compounds of the present invention are administered at a concentration of approximately 5 mg to approximately 50 mg per dose, approximately 5 mg to approximately 45 mg per dose, approximately 5 mg to approximately 40 mg per dose, approximately 5 mg to approximately 35 mg per dose, approximately 5 mg to approximately 30 mg per dose, approximately 5 mg to approximately 30 mg per dose, approximately 5 mg to approximately 25 mg per dose, approximately 5 mg to approximately 20 mg per dose, approximately 5 mg to approximately 15 mg per dose, approximately 5 mg to approximately 10 mg per dose, approximately 15 mg to approximately 50 mg per dose, approximately 15 mg to approximately 45 mg per dose, approximately 15 mg to approximately 40 mg per dose, approximately 15 mg to approximately 40 mg per dose, approximately 15 mg to approximately 35 mg per dose, approximately 15 mg to approximately 30 mg per dose, approximately 15 mg to approximately 25 mg per dose, approximately 15 mg to approximately 20 mg per dose, approximately 30 mg to approximately 50 mg per dose, approximately 35 mg to approximately 50 mg per dose, approximately 40 mg to approximately 50 mg per dose, or approximately 45 mg to approximately 50 mg per dose. As used herein, the term “approximately” means a concentration within 0.9 grams of the stated concentration values.

The composition may be administer once a day, at least twice a day, at least three times a day, at least four times a day, at least 5 times a day, or at least 6 times a day.

The invention is further described in the following examples, which do not limit the scope of the invention described in the claims.

### EXAMPLES

### Example 1

### Preparation of Asparagusic Acid and Derivatives

As the Applicant is an individual inventor, the most readily available source for asparagusic acid and its derivatives, Asparagus officinalis (asparagus), was used in these examples. However, the presence of asparagusic acid and its derivatives within asparagus is known and said compounds may be further isolated and/or synthesized according to known methods in the art.

In order to ensure proper preparation directly from asparagus, the asparagus must not be overheated or polymer sulfur compounds will be formed. (2). It was estimated that concentration of asparaptine to be at 10 mg per 50 grams of asparagus. (2). Assuming complete hydrolysis of the asparaptine, it was estimate that there is approximately 4.7 mg of asparagusic acid per 50 g of asparagus. The concentration of asparagusic acid isolated from asparagus is approximately 148 g per 500 g of asparagus, or 15 mg of asparagusic acid in 50 g of asparagus. (1) Asparagusic acid itself is not vary stable and undergoes rearrangement during cooking (boiling for 30 minutes) to yield 1,2,3-trithiane-5-carboxylic acid and 1,2-dithiacylopentene. (1) [CITE REF].

A patient diagnosed with Parkinson's disease and in need of treatment by the methods disclosed herein was administered compositions comprising the compounds disclosed herein. The patient had been diagnosis with Parkinson's disease by two medical doctors, the family doctor and a neurologist. He had taken L-Dopa for over a year. The patient informed his doctors that he was no longer taking L-Dopa. He took nothing for his Parkinson's Disease for almost a year before beginning to take asparagusic acid.

The test for relief of the shaking from Parkinson disease was the patient's hand writing skill. Prior to treatment with asparagusic acid the patient could not write his name. With treatment, the patient could easily write his name in a flowing easy manner. Normally the ability to write was diminished within 12 hours, but his varied.

The asparagus top third was cut from the stalk and weighed. 50 to 100 grams was used per treatment. The tops were steamed for a short period of time. This was typically done by placing the asparagus tops in a pie plate and covered. No water was added. After a short heating period, for example heating in a microwave for 44 seconds on high, the asparagus is “self” steamed from water in the plant and may substantially help in the formation of asparagusic acid from asparaptine. External steaming was done initially and it likewise resulted in a therapeutic effect. Excess heating will decompose asparagusic acid and will likely form a dihydro byproduct by splitting the disulfide linkage.

A preparation as described above was taken at least two hours after eating. The asparagus was consumed immediately after heating with salt and taken with simple non-caffeine liquids such as water and white wine. No further food was consumed for at least an hour. Consumption of food with the asparagusic acid preparation significantly diminished the effect. One exception was found. Hollandaise sauce made with a raw egg when eaten with slightly steamed asparagus also resulted in a therapeutic effect. It is noted that raw eggs contain many sulfur compounds.

When the patient had his dosage with dinner, the effects, as measured by the hand writing skill test noted above, were diminished substantially sometimes to no effect. This was repeated 14 times with similar results each time.

### Example 2

Asparagus tips were prepared with scrambled eggs but did not relieve shaking.

### Example 3

Egg nog was prepared with raw eggs and no asparagus and did not relieve shaking.

### Example 4

Asparagus soup was prepared with over 500 grams of asparagus and had no effect on shaking.

### Example 5

If the treatment is done with consumption of a caffeine drink, the effect is greatly diminished.

### Example 6

The patient had not eaten anything and had 3 cups of coffee prior to treatment. There was only a partial effect.

The patient consumed two and a half commercial glazed donuts two hours after treatment. The excess sugar produced an insulin event and relief from shaking was marginal.

### Example 7

The 1,2-ditholane ring in asparagusic acid is also found in lipoic acid. The presence of both functional groups and the 1,2-dithiolane ring, and the organic acid would suggest that lipoic acid would have a similar effect. The patient took a 250 mg tablet of lipoic acid in the same manner in regards to food consumption noted in Example 1 but there was no effect.

### REFERENCES CITED

(1) Mitchell et.al. Phytochemistry, 2014 January;97:5-10. doi: 10.1016/j .phytochem.2013.09.014. Epub 2013 October 4. By S. C. Mitchell and R. H. Waring entitled “Asparagusic Acid.”
(2) “Drug Metabolism and Disposition” volume 29 (4) Apr. 1, 2001 pages 539 -543 by S. C. Mitchell, titled “Food Idiosyncrasies: Beetroot and Asparagus”
(3) Nakabayashi et al. “Top-down targeted metabolomics reveals a sulfur-containing metabolite with inhibitory activity against angiotensin-converting enzyme in Asprargus officinalis.”J. of Nat Prod, 2015, 78(5):1179-83
(4) http://www.esrf.eu/home/news/general/content-news/general/copper-in-parkinsons-disease.html (2014)
(5) Squitti et.al. J, of Neurobiology of Aging, vol 35, sup 2, pp S40-S50. “Low Copper Diet as a preventive strategy for Alzheimer's Disease
(6) O. Demirkol, C. Adams and N. Ercal report in The J. of Agri Food Chem 2004, volume 52(26) page 8151-4. Titled “Biologically important thiols in various vegetables and fruits.”
(7) Wikipedia, Copper in Health Dec. 1, 2015.
(8) Wikipedia, Captoril, Dec. 1, 2015.
(9) Huheey, Inorganic Chemistry 3^{rd} Ed. Harper & Row, New York 1983, page 314.
(10) Scheiber, et.al. Progress in Neurobiology, 2014 volume 116 (5), pages 33-57 Titled “Metabolism and function of copper in brain.”
(11) Desai, et.al. American Journal of Clinical Nutrition, September 2008, volume 88 no.3 pages 8555-8585
(12) Berkeley Lab news release, November 2014, “Copper on the Brain.”
(13) Montes, et.al. Oxidative Medicine and Cellular Longevity, Volume 2014 (2014), Article ID 147251, 15 pages Titled: Copper and Copper Proteins in Parkinson's Disease.
(14) Bento et al. Acta Crystallogr D Biol Crystallogr, 2007 1(63, Part 2): 240-248
(15) Wang et al. Adv Nutr 2011, 2:129-37.
(16) Howell et al. Mol. Pharmacol. 2011, 77:887-94.

What is claimed is:

1. A method for treating a disease characterized by improper copper and/or sulfur concentrations in nerve cells of the central nervous system comprising; administering to a subject in need thereof an effective amount of a composition comprising one or more compounds selected from the following: 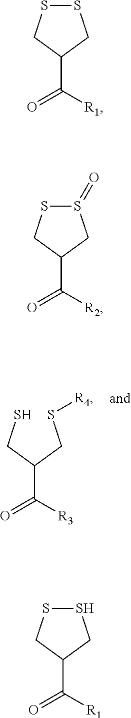
wherein R1 is H, CH₃, OH, or OCH₃
R2 is H, CH₃, OH, or O CH₃
R3 is H, CH₃, OH, or O CH₃ and
R4 is H, CH₃, OCH₃, or
a condensation product of the one or more compounds with an amino acid, or both.

2. The method of claim 1, wherein the subject is suffering from nerve cell malfunction and/or death in the substantia nigra region of the brain.

3. The method of claim 1, wherein the subject has Parkinson's disease.

4. The method of claim 1, wherein the composition is administered at a concentration of approximately 15 to 30 mg per dose.

5. The method of claim 3, wherein the composition is administered at least twice, at least three times, or at least four times a day.

6. The method of claim 1, wherein the compound is administered orally.

7. The method of claim 1, wherein the compound is asparagusic acid, asparagusic acid methyl ester, asparagusic ethyl ester, asparagusic acid glucoside; asparagusic acid S-oxide, asparagusic acid S-oxide methyl ester, dihydroasparagusic acid, or S-acetyldihydroasparagusic acid.

8. The method of claim 1, wherein the compound is asparagusic acid.

9. The method of claim 1, wherein the amino acid that forms the condensation product with the one or more compounds is arginine.

10. The method of claim 1, wherein the condensation product is asparaptine.

11. A method for restoring impaired motor function associated with neurological diseases comprising; administering to a subject in need thereof an effective amount of a composition comprising one or more compounds selected from the following: 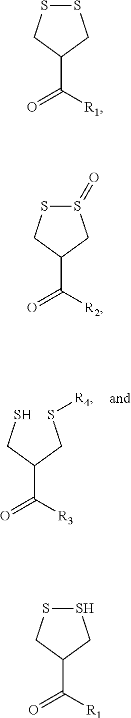
wherein R1 is H, CH₃, OH, or OCH₃,
R2 is H, CH₃, OH, or O CH₃,
R3 is H, CH₃, OH, or O CH₃ and,
R4 is H, CH₃, OCH₃; or
a condensation product of the one or more compounds with an amino acid, or both.

12. The method of claim 7, wherein the subject suffers from Parkinson's disease, Menke's disease, Wilson's disease, or Alzheimer's disease.

13. The method of claim 7, wherein the subject suffers from one or more of tremor, bradykinesia, rigidity, and postural instability.

14. The method of claim 7, wherein the composition is administered at a concentration of approximately 15 to 30 mg per dose.

15. The method of claim 7, wherein the composition is administered at least twice, at least three times, or at least four times a day.

16. The method of claim 7, wherein the composition is administered orally.

17. The method of claim 7, wherein the compound is asparagusic acid, asparagusic acid methyl ester, asparagusic ethyl ester, asparagusic acid glucoside; asparagusic acid S-oxide, asparagusic acid S-oxide methyl ester, dihydroasparagusic acid, or S-acetyldihydroasparagusic acid.

18. The method of claim 14, wherein the compound is asparagusic acid.

19. The method of claim 7, wherein the amino acid that forms the condensation product with the one or more compounds is arginine.

20. The method of claim 7, wherein the condensation product is asparaptine.

21. A method of increasing copper and/or sulfur concentration in nerve cells and/or restoring or maintaining proper copper and/or sulfur homeostasis in nerve cells, comprising:
administering to a subject in need thereof an effective amount of a composition comprising one or more compounds selected from the following: 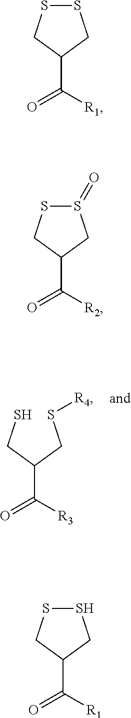
wherein R1 is H, CH₃, OH, or OCH₃,
R2 is H, CH₃, OH, or 0 CH₃,
R3 is H, CH₃, OH, or 0 CH₃ and,
R4 is H, CH₃, OCH₃; or
a condensation product of the one or more compounds with an amino acid, or both.

22. The method of claim 20, wherein the subject suffers from Parkinson's disease.

23. The method of claim 20, wherein the subject suffers from one or more of tremor, bradykinesia, rigidity, and postural instability.

24. The method of claim 20, wherein the composition is administered at a concentration of approximately 15 to 30 mg per dose.

25. The method of claim 20, wherein the composition is administered at least twice, at least three times, or at least four times a day.

26. The method of claim 20, wherein the compound is administered orally.

27. The method of claim 20, wherein the compound is asparagusic acid, asparagusic acid methyl ester, asparagusic ethyl ester, asparagusic acid glucoside; asparagusic acid S-oxide, asparagusic acid S-oxide methyl ester, dihydroasparagusic acid, or S-acetyldihydroasparagusic acid.

28. The method of claim 26, wherein the compound is asparagusic acid.

29. The method of claim 20, wherein the amino acid that forms the condensation product with the one or more compounds is arginine.

30. The method of claim 20, wherein the condensation product is asparaptine.
